(19) United States
(12) Patent Application Publication
(10) Pub. No.: US 2005/0176826 A1
(43) Pub. Date: Aug. 11, 2005

(54) Use of asc-1 inhibitors to treat neurological and psychiatric disorders

(75) Inventors: Christian Thomsen, Stroby (DK); Lone Helboe, Frederiksberg (DK); Jan Egebjerg Jensen, Arhus N. (DK)

Correspondence Address: DARBY & DARBY P.C., P. O. BOX 5257, NEW YORK, NY, 10150-5257, US

(73) Assignee: H. Lundbeck A/S, Copenhagen- Valby (DK)

(21) Appl. No.: 10/506,087

(22) PCT Filed: Mar. 12, 2003
(86) PCT No.: PCT/DK03/00154

Related U.S. Application Data
(60) Provisional application No. 60/365,070, filed on Mar. 15, 2002.

(30) Foreign Application Priority Data
Mar. 15, 2002 (DK) 2002 00417

(51) Int. Cl. A61K 31/198; A61K 51/00

(52) U.S. Cl. 514/561; 424/1.11; 514/562

(57) ABSTRACT

The present invention relates to the identification and use of compounds that are inhibitors of the alanine-serine-cysteine transporter 1 (asc-1). This includes an assay for the identification of compounds that are inhibitors of asc-1, as well as pharmaceutical compositions comprising these compounds. The invention also comprises a method for the use of these compositions for the treatment, alleviation or amelioration of memory and attention deficits resulting from but not limited to Alzheimer's disease, Parkinson's disease, trauma and stroke, and for enhancement of learning and memory ability in a human not suffering from any neurological disorders. Finally, the invention comprises methods for use of the compositions for alleviation or amelioration of conditions in which there is altered glutamatergic or dopaminergic neurotransmission such as schizophrenia, Parkinson's disease, epilepsy, depression, obsessive compulsive disorders and bipolar disorders.

### FIELD OF INVENTION

The present invention provides methods for the identification and use of compounds that are inhibitors of the alanine-serine-cysteine transporter 1 (asc-1). These methods include the use of such inhibitors of asc-1 for the preparation of a pharmaceutically acceptable composition for treatment, alleviation or amelioration of memory and attention deficits that result from Alzheimer's disease, Parkinson's disease, trauma and stroke. The composition may also be used to enhance the function of normal excitable tissue, such as for facilitating learning and memory. Furthermore, the composition can be used for alleviation or amelioration of conditions in which there are altered glutamatergic or dopaminergic neurotransmission such as schizophrenia, Parkinson's disease, epilepsy, depression, obsessive compulsive disorders and bipolar disorders. The present invention also embraces pharmaceutical compositions comprising these compounds and methods of using the compounds and their pharmaceutical compositions.

### BACKGROUND OF THE INVENTION

Dopamine and glutamate are neurotransmitters that are very important for the normal function of the central nervous system. Accordingly, dysfunction in these neurotransmitter systems have been associated with a number of neurological and psychiatric disorders including Alzheimer's disease, Parkinson's disease, schizophrenia, epilepsy, depression, obsessive compulsive disorders and bipolar disorders (Parsons et al., Drug News Perspect. 1998, 11, 523-533; Goff and Coyle, Am J Psychiatry 2001, 158, 1367-1377). It has now become evident that these two systems are highly interconnected and that blockade of a receptor for the glutamate system can affect the release of the transmitter dopamine and vice versa (for reviews see: Goff and Coyle, Am J Psychiatry 2001, 158, 1367-1377; Whitton, Neurosci Biobehav Rev, 1997, 21(4), 481-488; Jentsch and Roth, Neuropsychopharmacology, 1999, 20, 201-205). For example, the administration of non-competitive NMDA receptor antagonists is associated with a profound increase in dopamine transmission in different brain areas including forebrain areas and ventral tegmental area (Takahata and Moghaddam, J Neurochem 1998, 71, 1443-1449; Goff and Coyle, Am J Psychiatry 2001, 158, 1367-1377; Whitton, Neurosci Biobehav Rev, 1997, 21(4), 481-488; Jentsch and Roth, Neuropsychopharmacology 1999, 20, 201-205). Conversely, blockade of dopaminergic transmission with dopamine D2 antagonists such as haloperidol and clozapine has been shown to enhance glutamatergic transmission by enhancing the function of the NMDA receptor at clinically relevant concentrations (Banerjee et al. Neuroreport, 1995, 6, 2500-2504). Accordingly, augmenting NMDA receptor function in specific areas of the central nervous system may be beneficial for affective disorders, including depression, obsessive compulsive disorders, bipolar disorders, psychosis and schizophrenia for which dopamine has a central role (McDougle J Clin Psychiatry 1997, 58, 11-17; Naranjo et al. Prog Neuropsychopharmacol Biol Psychiatry 2001, 25, 781-823).

The NMDA receptor is very well established to be pivotal for memory and learning processes (Parsons et al. Drug News Perspect. 1998, 11, 523-533; Danysz and Parsons Pharmacol Rev 1998, 50, 597-664). The functioning of the NMDA receptor requires the activation of both the agonist binding site for glutamate and the allosteric co-agonist site which is strychnine insensitive and activated by glycine and D-serine (Kleckner and Dingledine, Science 1988, 241, 835-837; McBain et al, Mol Pharmacol 1989, 36, 556-565; Danysz and Parsons Pharmacol Rev 1998, 50, 597-664). Activation of the D-serine-sensitive modulatory site on the NMDA receptor has been shown to be a prerequisite for induction of long term potentiation (Bashir et al. Neurosci Lett. 1990, 108, 261-266), an in vitro correlate of memory and learning.

Furthermore, the cognitive deficits associated with psychiatric disorders such as schizophrenia have been shown to be alleviated by oral treatment with D-serine (Tsai et al. Biol Psychiatry 1998, 44, 1081-1089). Thus, agents that cause an increase in glycine or D-serine concentrations at locations where the NMDA receptor is expressed are expected to be general memory enhancing agents both in humans suffering from a pathological deficit and also in normal humans. Furthermore, based on the above, such agents are expected to be effective against cognitive dysfunction associated with neurological diseases including but not limited to Parkinson's and Alzheimer's disease or associated with psychiatric disorders such as schizophrenia.

A beneficial effect of augmented NMDA receptor function for the indication of epilepsy may seem controversial because direct activation of the NMDA receptor is known to cause convulsions and NMDA antagonists are generally anticonvulsants (Meldrum et al. Epilepsy Res. 1999, 36, 189-204). However, at the level of the neuronal circuitry stimulating NMDA receptors may cause net inhibition if the activated neurons are inhibitory and projects to primary major excitatory pathways (Olney et al. J Psychiatr Res. 1999, 33, 523-533). Furthermore, at the molecular level the NMDA receptor has been shown to be coupled to activation of a potassium channel indicating that the receptor may be inhibitory in certain synapses (Isaacson and Murphy Neuron 2001, 31, 1027-1034). Positive allosteric modulators acting at the strychnine-insensitive site at the NMDA receptor such as D-serine and D-cycloserine have indeed been shown to be anticonvulsants in several studies (Peterson Eur J Pharmacol 1991, 199, 341-348; Peterson and Schwade, Epilepsy Res, 1993, 15, 141-148; Loscher et al. Br J Pharmacol 1994, 112, 97-106). These effects were blocked by the specific antagonist at this site, 7-chlorokynurenic acid (Loscher et al. Br J Pharmacol. 1994, 112, 97-106; Peterson Eur J Pharmacol. 1991, 199, 341-348). Furthermore, D-serine (but not L-serine indicating stereospecificity) potentiated the anticonvulsant effects of established antiepileptics (Peterson Eur J Pharmacol. 1991, 199, 341-348). Thus, positive allosteric modulation of the NMDA receptor using such agonists at the strychnine-insensitive site is a novel treatment paradigm for seizure disorders, including epilepsy and may be used alone or in combination with established anticonvulsant drugs.

A leading hypothesis proposes that deficits in NMDA receptor-mediated neurotransmission is an underlying mechanism for the pathophysiology of schizophrenia (Jentsch and Roth Neuropsychopharmacology 1999, 20, 201-205; Olney et al. J. Psychiatr Res. 1999, 33, 523-533). The foundation of this hypothesis derives from the clinical effects of NMDA receptor antagonists, such as phencyclidine (PCP) and ketamine that induce schizophrenic-like symptoms in man (Jentsch and Roth Neuropsychopharmacology 1999, 20, 201-205; Olney et al. J. Psychiatr Res. 1999, 33, 523-533). Augmenting NMDA receptor function in a “non-toxic” manner could provide a treatment strategy for schizophrenia In preclinical studies, glycine and D-serine reverse the behavioural effects of PCP in rodents (Contreras Neuropharmacology 1990, 29, 291-293; Javitt et al. Neuropsychopharmacology 1997, 17, 202-204; Tanii et al. J Pharmacol Exp Ther. 1994, 269, 1040-1048; Nilsson et al. J Neural Transm 1997, 104, 1195-1205). Based upon the observations that L-glycine and D-serine are effective in such animal models related to NMDA function, it can be concluded that glycine sites are not saturated under normal physiological conditions. More strikingly, small clinical studies have assessed the therapeutic potential of glycine site agonists of the NMDA receptor, such as glycine, D-serine and D-cycloserine (Javitt et al. Am J Psychiatry 1994, 151, 1234-1236; Heresco-Levy et al. Br J Psychiatry 1996, 196, 610-617; Tsai et al., 1998). The results of these studies indicate that this type of compounds may reduce negative symptoms and alleviate cognitive deficits in schizophrenia patients (Javitt et al. Am J Psychiatry 1994, 151, 1234-1236; Heresco-Levy et al. Br J Psychiatry 1996, 196, 610-617; Tsai et al. Biol Psychiatry 1998, 44, 1081-1089). In addition, a study suggests a beneficial effect against positive symptoms (Tsai et al. Biol Psychiatry 1998, 44, 1081-1089).

Until now, treatment strategies for schizophrenia have focused on agents that potentiate NMDA receptor-mediated neurotransmission by binding to the NMDA-associated glycine binding site. However, the clinical use of glycine and D-serine is hampered by the fact that large doses must be given to penetrate the blood-brain barrier. Furthermore, efficient uptake systems for these amino acids are likely to limit their therapeutic effectiveness. Indeed, the reason that endogenous glycine does not saturate NMDA receptors under physiological conditions is that such receptors are protected from high extracellular levels by the activity of glycine transporters that to some extent are co-localised with NMDA receptors (Smith et al. Neuron 1992, 8, 927-935; Danysz and Parsons Pharmacol Rev 1998, 50(4), 597-664). This has formed the rationale for current drug discovery projects targeting glycine transporters such as GlyT-1 for developing novel antipsychotics based on the glutamate hypo-function hypothesis of schizophrenia. However, the inhibition of D-serine transport may be more favorable than the inhibition of L-glycine transport, since a) the distribution and developmental pattern of D-serine co-localise with the NMDA receptor, while the distribution of L-glycine is more ubiquitous (Schell et al. J Neurosci 1997, 17(5), 1604-1615; Hashimoto et al. Eur J Neurosci 1995, 7, 1657-1663) and b) D-serine is a 3-4 fold more potent co-agonist than glycine at the allosteric site on the NMDA receptor (Matsui et al. J Neurochem 1995, 65, 454-458), and more specifically because L-glycine also interacts with the strychnine-sensitive glycine receptor which is implicated in control of movements (Betz et al. Ann N Y Acad Sci 1999, 868, 667-676).

The central nervous system contains multiple amino acid transport systems, including systems “Gly”, “A”, “L” that are specialised for uptake of glycine, alanine and leucine, respectively, and furthermore, “ASC” which is specialised for uptake of alanine, serine and cysteine (Christensen Physiol Rev 1990, 70, 43-77; Hashimoto and Oka Prog Neurobiol 1997, 52, 325-353). Transport of both isomers of serine is in general considered to be mediated via system ASC despite the fact that transport may also occur through system L (Christensen Physiol Rev. 1990, 70, 43-77; Hashimoto and Oka Prog Neurobiol. 1997, 52, 325-353). Two ASC-like transporters have recently been cloned and have been termed ASCT1 (Arriza et al. J Biol Chem, 1993, 268(21), 15329-15332) and ASCT2 (Utsunomiya-Tate et al. J Biol Chem. 1996, 271(25), 14883-14890). Studies with these cloned transporters have confirmed that ASC-family transporters show highest affinity for L-alanine, along with high affinity for L-cysteine and L-serine, and stereoselectivity for Lo over D-amino acids. Based on the relatively low affinity of these transporters for D-amino acids, the presence of additional systems with specificity for D-amino acids, including D-serine could be postulated to maintain levels of D-serine in the brain relatively low (Hashimoto et al. Neuroscience 1995, 66, 635-643).

Recently, the cloning and characterisation of a novel Na⁺-independent alanine-serine-cysteine transporter (asc-1) has been reported (Nakauchi et al. Neurosci Lett 2000, 287, 231-235). A second member of this transporter family has also recently been cloned and termed asc-2 (Chairoungdua et al. J Biol Chem 2001, 276(52), 49390-49399). This transporter showed preference for small neutral amino acids similar to asc-1. However, although it has been shown that asc-1 RNA is expressed in total brain extract (Nakauchi et al. Neurosci Lett. 2000, 287, 231-235), the tissue distribution of the asc-1 protein has not been reported, and therefore the significance of this transporter with regard to physiology and disease in the body has not been determined. Asc-2 was not detected in the brain (Chairoungdua et al. J Biol Chem. 2001, 276(52), 49390-49399), which implies that asc-2 is not a target for diseases related to the central nervous system.

Limitations in the use of D-serine for treatment of CNS diseases as described above are, that large doses must be administered, in order for sufficient D-serine to pass the blood brain barrier and furthermore, that transport systems exist in the brain, that will prevent increases in the concentration of exogenously administered D-serine at critical sites in the brain. Thus, alternative ways must be found, in order to ameliorate D-serine levels at critical locations of the brain.

### DESCRIPTION OF THE INVENTION

It has now been found, as described in the present invention, that asc-1 inhibitors will have the potential of ameliorating D-serine levels at sites in the brain where NMDA receptors are expressed. Accordingly, this application relates to the use of Na⁺-independent D-serine transport inhibitors, in particular inhibitors of asc-1, to ameliorate NMDA receptor-mediated neurotransmission. More specifically, the present invention relates to the use of asc-1 inhibitors for the treatment of schizophrenia, psychosis, Parkinson's disease, depression, obsessive compulsive disorder, an anxiety disorder, a bipolar disorder, epilepsy, or memory and attention deficits resulting from Alzheimer's disease, Parkinson's disease, trauma and stroke, as well as for enhancement of learning and memory.

Claimed is a pharmaceutical composition characterised in that it comprises a therapeutically effective amount of an inhibitor of the asc-1 transporter, as well as a relevant pharmaceutically acceptable carrier. A therapeutically effective amount of an asc-1 inhibitor is the amount of inhibitor needed for treatment of a certain condition Treatment in the sense of this invention comprises treatment, alleviation and amelioration of symptoms and/or complete or partial inhibition of progression of the disease.

The invention additionally comprise the use of an inhibitor of asc-1 for the manufacture of a medicament for the treatment of schizophrenia, Parkinson's disease, depression, obsessive compulsive disorder, an anxiety disorder, a bipolar disorder, seizure disorders, epilepsy, memory and attention deficits resulting from Alzheimer's disease, Parkinson's disease, trauma or stroke, in a human suffering from such a disease. In particular, in schizophrenic patients, the negative symptoms may be reduced and the cognitive deficits may be alleviated. In patients suffering from seizure disorders and epilepsy, an asc-1 inhibitor may be anticonvulsant, and may be used alone or in combination with established anticonvulsant drugs. Due to the effects of asc-1 inhibition on D-serine mediated NMDA receptor signalling, the asc-1 pharmaceutical compositions may be used to treat the cognitive and memory deficits observed in the above mentioned diseases.

Furthermore, the asc-1 inhibitor may be used to manufacture a medicament useful for enhancing the function of normal or abnormal excitable tissue, including enhancing associative learning and memory.

The invention also provide methods useful for the identification of asc-1 inhibitors, by use of the enclosed assays where the ability of a compound to inhibit the transport of D-serine across cortical membranes or across membranes from HEK293 cells expressing human asc-1 protein is observed.

Methods for synthesis and screening of such compounds based upon the described assays are apparent to practitioners skilled in the art.

Pharmaceutical compositions comprising such asc-1 inhibitors in a non-toxic amount and a pharmaceutically acceptable carrier, made for the treatment of diseases in the CNS are enclosed. In a preferred embodiment of the invention, the pharmaceutical composition comprise a quantity of active compound in a unit dose of preparation that may be varied or adjusted from about 0.1 mg to 1000 mg, more preferably from about 1 mg to 300 mg, according to the particular application.

Thus, the present application claims use of asc-1 transport inhibitors, at doses sufficient to elevate brain D-serine/L-glycine levels, for the treatment of neurological and psychiatric disorders as defined in the present invention. In a further embodiment, the invention relates to the use of such asc-1 inhibitors to enhance the function of normal or abnormal excitable tissue.

The invention is partly based on the discovery that asc-1 is located in areas of the brain also known to contain NMDA receptors and D-serine. This is the first time the expression of a specific transport protein (asc-1) with high affinity for D-serine have been demonstrated to be co-localised with the NMDA receptor and with D-serine in the brain. Furthermore, it has been found, that a large component of D-serine transport across rat cortical synaptosomal membranes is Na⁺ independent and has a substrate specificity, that is reminiscent of the cloned asc-1. The substrate specificity of asc-1 was compared to that of brain cells by comparing the effects of 20 natural amino acids for inhibiting [³H]D-serine uptake in HEK293 cells expressing the cloned asc-1 and rat cortical synaptosomes, respectively. There was a significant correlation between the respective pKᵢ's (human asc-1 versus rat cortical membranes: P<0.0001, r²=0.57, F=32, n=26, slope=0.94) suggesting that the D-serine-sensitive Na⁺-independent transporter present in rat brain P2 synaptosomes is of the “asc-1” type.

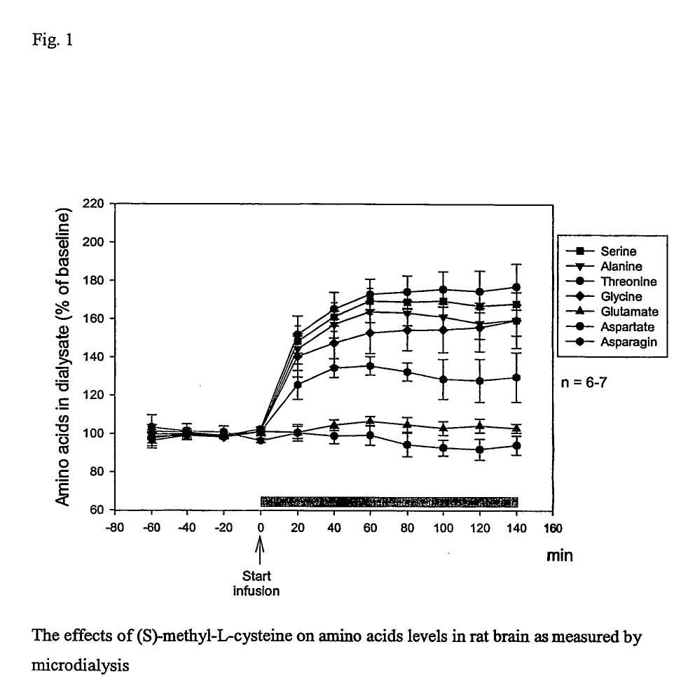

Consequently, it was found, according to the present invention, that inhibition of the asc-1 D-serine transporter, will result in increased D-serine concentrations in discrete areas of the brain, including areas where asc-1 is co-localised with the NMDA receptor. This was demonstrated by using (S)-methyl-L-cysteine which we show is a potent and selective inhibitor of [³]D-serine uptake in rat cortical synaptosomes, rat cerebellar synaptosomes and in HEK293 cells expressing the human asc-1 transporter. (S)-Methyl-L-cysteine has previously been shown to be a weak inhibitor (81% inhibition at 5 mM corresponding to an IC₅₀˜1.2 mM) of System A transporters as measured by inhibition of [³H]AIB transport into cultured rat hepatocytes (Bracy et al., J Biol Chem 1986, 261, 1514-1520). In the present invention we showed that (S)-methyl-L-cysteine is much more potent at inhibiting [³H]D-serine transport into HEK293 cells expressing human asc-1 (Kᵢ=62±15 μM) or into rat cortical membranes (Kᵢ=6.6±1.8 μM). Furthermore, (S)-methyl-L-cysteine does not block the transport of other amino acids usually implicated in psychosis such as serotonin (Kᵢ>1 mM), noradrenaline (Kᵢ>1 mM), dopamine (Kᵢ>1 mM) or glutamate (Kᵢ>1 mM). In addition, (S)-methyl-L-cysteine did not block the glycine transporter (GlyT-1B) (Kᵢ>100 μM). When this asc-1 inhibitor is infused via the microdialysis probe into rat brain a marked increase in the levels of serine, alanine, threonine and glycine was observed (FIG. 1). These amino acids are known substrates for asc-1 (Fukasawa et al., 2000, J Biol Chem 275, 9690-9698; Nakauchi et al. Neurosci Lett 2000, 287, 231-235) and the observed increases are in accordance with the perception that the transporters operates in an exchange mode (Fukasawa et al., 2000, J Biol Chem 275, 9690-9698). Amino acids which are not substrates for asc-1, such as glutamate and aspartate, were not affected (FIG. 1) indicating that the effect was specific for asc-1 inhibition. Furthermore, serine, alanine and threonine are not substrates for glycine transporters (Kim et al., 1994, Liu et al., 1993, J Biol. Chem 268, 22802-22808) suggesting that glycine transporter blockade is unlikely to mediate the effects observed.

These discoveries in combination with the amino acid uptake pharmacology of asc-1 indicates, that selective asc-1 inhibitors would produce behavioural and neurochemical effects similar to those produced by large doses of D-serine, glycine, or glycine transport inhibitors.

These effects include potentiation of NMDA receptor-mediated neurotransmission and reversal of PCP-induced behavioural and neurochemical effects. Accordingly, asc-1 inhibitors will alleviate cognitive dysfunction related to schizophrenia, Alzheimer's disease, Parkinson's disease, trauma and stroke. Asc-1 inhibitors will also be efficacious in conditions in which there is altered glutamatergic or dopaminergic neurotransmission such as schizophrenia (both against negative and positive symptoms), Parkinson's disease, depression, obsessive compulsive disorders and bipolar disorders. Furthermore, asc-1 inhibitors should be effective for treating seizure disorders including epilepsy, alone or in combination with established anticonvulsant drugs. Agents that potentiate NMDA receptor-mediated neurotransmission in vivo have shown effectiveness in the treatment of persistent negative and cognitive symptoms of schizophrenia. Finally, based on the findings of the invention it can be expected that applying selective inhibitors of asc-1 to a mammal will lead to an enhancement of the function of normal or abnormal excitable tissue, resulting in the enhancement of associative learning and memory.

A preferred aspect of the invention relates to prevention or treatment wherein a dose of an asc-1 inhibitor is administered prophylactically for preventing a progress of the condition or of any symptom of the condition (e.g. for patients at risk of suffering from a stroke).

For the administration to an individual suffering from one of the above mentioned diseases, the asc-1 inhibitor may be formulated into a pharmaceutical composition containing the inhibitor and optionally one or more pharmaceutically acceptable excipients.

In another preferred embodiment of the invention, the quantity of the active compound in the pharmaceutical composition, in a unit dose of preparation may be varied or adjusted from about 0.1 mg to 1000 mg, more preferably from about 1 mg to 300 mg, according to the particular application.

The asc-1 protein is widely distributed in the brain and is also located in areas with high expression of NMDA receptors, (e.g. cerebral cortex, hippocampus, amygdala, nucleus accumbens, substantia nigra—for a more detailed description of the expression pattern in the brain see below). Furthermore, it has been found that a large component of [³H]D-serine uptake into rat cortical synaptosomes is Na⁺-independent (i.e. the maximal velocity (Vₘₐₓ for [³H]D-serine uptake in rat cortical membranes is ˜20-25% lower in the presence of 120 mM Na⁺-ions as compared to the Vₘₐₓ measured in the absence of added Na⁺-ions) and has a substrate specificity reminiscent of the cloned asc-1 indicating that asc-1 is a major contributor to overall clearance of D-serine in brain. This was among other things demonstrated by comparing the effects of 20 natural amino acids for inhibiting [³H]D-serine uptake in HEK293 cells expressing the cloned asc-1 and rat cortical synaptosomes, respectively, using the protocols detailed in this application. In particular, an effective inhibition was observed by amino acids, L-alanine, L-cysteine, L-glycine, L-serine and L-threonine in both types of assays. Less effective, but significant inhibition was observed in both assays by using L-asparagine, L-histidine, L-isoleucine, L-leucine, L-methionine, L-phenylalanine, L-tyrosine and L-valine. Inactive amino acids included L-aspartate, L-arginine, L-cystine, L-glutamate, L-glutamine, L-lysine and L-proline. Quantitatively similar results were obtained with the corresponding D-isomers. A highly significant correlation between the respective pKᵢ'for inhibiting [³H]D-serine uptake into HEK293 cells expressing human asc-1 versus uptake into rat cortical membranes was found: P<0.0001, r²=0.57, F=32, n=26, slope=0.94. A previous application has described a putative novel transporter for [³H]D-serine in rat brain which is characterised by its insensitivity to L-alanine. Indeed, in this application (Javitt, WO 01/08676 A1) 30 mM L-alanine was included in the assay conditions. However, this concentration is more than sufficient to completely block uptake via the asc system, including asc-1. Furthermore, L-alanine completely blocks [³H]D-serine uptake into rat cortical membranes which therefore does not suggest the presence of L-alanine-insensitive D-serine transporters. Accordingly, the asc-1 transporter described in the present application is clearly distinct from the uptake system described in the WO 01/08676 application.

Experimentals

Cloning and Expression of Human asc-1:

The cDNA encoding the human Na⁺-independent transporter asc-1 (Nakauchi et al. Neurosci Lett, 2000, 287, 231-235) and the human type II membrane glycoprotein, 4F2 heavy chain were isolated using standard RT-PCR procedures on human brain RNA. The fragments were cloned into the mammalian expression vector pCI/neo (Promega Corporation) and co-transfected into HEK293 cells (American Type Culture Collection #CRL 1573) using lipofectamine. Uptake was determined 2-4 days after the transfection.

Localisation of asc-1 as Determined by Immunohistochemistry:

A specific polyclonal antibody was raised against the peptide sequence PSPLPITDKPLKTQC located in the intracellular C-terminal domain of the transporter. The peptide was conjugated to keyhole limpet hemocyanine prior to immunization of New Zealand white rabbits. In Western blot analysis, the antiserum recognised a 40 kDa protein band in CHO-K1 cells (American Type Culture collection #CCL-61) transfected with the murine Asc-1. No bands were detected in untransfected control cells.

Adult male NMRI mice (M&B, Ry, DK) were fixed transcardially with 4% paraformaldehyde and the brains were dissected out. The brains were cryoprotected in 30% sucrose and 40 μm frontal cryosections were prepared and processed for immunohistochernistry. The sections were incubated overnight at 4° C. with the asc-1 antiserum. This was followed by incubation for 1 hour with biotin-labelled anti rabbit antibodies (DAKO) and horseradish peroxidase-conjugated streptavidin-biotin (Vector Laboratories). Imnunoreactivity was visualised with 0.05% diaminobenzidine and 0.01% H₂O₂. Prejimune serum and preabsorbed antiserum served as controls and did not result in any staining.

Asc-1-immunoreactivity (Asc-1-ir) was widely distributed throughout the mouse brain. Asc-ir was observed as punctuate staining consistent with varicosities matching neuronal cell bodies and dendritic fields. In few instances, staining of perikarya was observed. Inmunostaining in either glial cell bodies or perivascular sites was never observed.

The cerebral cortex was moderately labelled and appeared layered with the strongest signal in layers III and V. A prominent Asc-1-ir was observed in cingulate and retrosplenial cortices.

Medial septum showed a strong labelling and lateral septum weak Asc-1-ir. In the basal ganglia, globus pallidus exhibited an intense immunostaining with a particular strong staining in the medial part located in the ventral region of the internal capsule. Moderate and weak Asc-1-ir was present in nucleus accumbens and caudate putamen, respectively. The bed nucleus of stria terminalis was moderately stained. A moderate Asc-1-ir was seen in all amygdala nuclei with the strongest intensity in the medial areas.

In the hippocampus, an intense immunostaining was found in outer pyramidal cells of CA1, CA2, CA3 and hilus of the dentate gyrus. Moderate Asc-1-ir was present in stratum oriens and stratum radiatum moleculare. The molecular layer of the dentate gyrus was moderately stained and the granule cell layer was unstained. An intense Asc-1-ir was distributed throughout the hypothalamus in both medial and lateral areas, and including the external layer of the median eminence. No labelling of specific neuronal hypothalamic areas was distinguished.

Many thalamic areas showed Asc-1-ir, including lateral thalamic nuclei, lateral geniculate body, reticulate nuclei, paraventricular nucleus, centrolateral and centromedial thalamic nuclei, lateral habenula

Prominent Asc-1-ir was present in the brain stem. Areas with intense immunostaining include superficial layer of superior colliculus, supramammillary nucleus and also medial and lateral nuclei, the area surrounding the pyramidal tract corresponding to nuclei of trapezoid body, superior olive, ventral cochlear nucleus, lateral reticular formation, dorsal tegmental nuclei, hypoglossal nucleus, medial parabrachial nucleus, pontine nucleus, dorsal raphe. Moderate or weak staining was detected in periaqueductal grey, substantia nigra and nucleus of solitary tract.

An intense Asc-1-ir was present in the cerebellum mainly in the molecular layer including the Purkinje cells. Weak staining was observed in the granule layer. We observed expresion of the D-serine transporter asc-1 in many of the same areas as was described for D-serine by Schell (Schell et al. J Neurosci 1997, 17(5), 1604-1615). High levels of D-serine are found in the cerebral cortex, hippocampus, striatum, and to a lesser extent in the limbic forebrain, diencephalon and midbrain. Likewise, we find asc-1 highly expressed in these areas. However, we also find intense immunostaining for asc-1 in areas of low D-serine abundance such as the hypothalamus and the brain stem. But since Asc-1 also transports amino acids other than D-serine, this pattern of distribution may reflect that of other substrates, e.g. glycine.

Measurements of Na⁺-Independent [³H]D-Serine Uptake

Into cortical membranes: Cortex from male Wistar rats (150-200 g) was homogenized in 0.40 M sucrose and centrifuged at 1000×g for 10 min. The pellet was discarded and the supernatant was centrifuged at 40.000×g for 20 min and resuspended in assay buffer: 120 mM cholinechloride, 1.5 mM KCl, 1.2 mM CaCl₂, 1.2 MM MgSO₄, 1.2 mM KH₂PO₄ 10 mM D-glucose, 25 mM triethylammonium bicarbonate, 10 mM HEPES. Test compounds and tissue (1 mg orig. tissue/well) were added to 96 well plates and incubated with [³H]-D-serine (specific activity=26.8 Ci/mmol, PerkinElmer, Cambridge, U.K.) (100 nM final concentration) for 5 min at 25° C. Samples were filtered on Unifilter GF/B glass fiber (Packard Biosciences, Meriden, Conn., USA) and washed with 3×0.25 mL assay buffer. Measurements of [³H]-D-serine uptake into whole HEK293 cells expressing human asc-1 were performed in 96 well plates with similar conditions except that the cells were incubated for 15 min with radioligand and washed by dipping twice in cold assay buffer following incubation with radioligand. Accumulated radioactivity was extracted from the cells by adding 200 μL scintillation fluid/well (Ultima Gold, Packard Biosciences) and the plates were counted in a Microplates Scintillation Counter (Packard Biosciences). The D-serine uptake in samples containing test compounds was compared to controls without added compound or controls where transport via the asc-1 was inhibited by addition of e.g. 30 mM L-alanine.

In separate experiments, asc-1 mediated uptake was measured using [³⁵S]-L-cysteine (0.5×106 DPM/well, specific activity>1000 Ci/mmol, Amersham, Buckinghamshire, UK) as radioligand in place of [³H]-D-serine. All other experimental details were as described for [³H]-D-serine uptake experiments in asc-1/HEK293 cells.

When referring to asc-1 in connection with transfected cell lines, assays and screening procedures for the purpose of identification of asc-1 inhibitors, the term asc-1 implies the protein and posttranslational modified forms as described by Nakauchi (Nakauchi et al. Neurosci Let. 2000, 287, 231-235). Furthermore, in the same context as above asc-1 also includes, but is not limited to, naturally occurring proteins originating from splice variants and polymorphisms of the asc-1 gene. Furthermore, asc-1 in the definition of the invention includes peptide fragments of asc-1, asc-1 peptides with point mutations, as well as asc-1 protein/peptide fragments with high sequence identity to natural asc-1. High sequence identity in the meaning of the invention means that included are asc-1 peptide fragments/proteins that at the amino acid level exhibit identity within the range of 60%, 70%, 80%, 90% or most preferred at least 95% to the published sequence.

Measurements of Amino Acid Uptake

Measurements of [³H]-glycine uptake into CHO cells expressing human GlyT-1B were performed in 96 well plates using 1 μCi [³H]-glycine/well. Cells were plated 2 days before the experiment and washed twice with assay buffer (composition: 150 mM NaCl, 10 mM glucose, 2.5 mM KCl, 1 mM CaCl₂, 2.5 mM MgSO₄, 10 mM HEPES, pH 7.4). Test compounds were added 10 min before radioligand and cells were incubated for a further 15 min at 37° C. Cells were washed as described for [3 H]-D-serine uptake into asc-1 cells. Non-specific uptake was defined as uptake in the presence of 100 μM N-methyl-N-(phenyl-trifluoromethylphenoxy)propan-1-yl-glycine.

Inhibition of serotonin (5-HT), dopamine (DA) and noradrenaline (NA) uptake in vitro was measured in rat brain synaptosomes using a modification of a previously described protocol (Bøgesø et al.,, J Med Chem 1985, 28, 1817-1828). In brief, tritium labelled amines were used to measure uptake into synaptosomes from whole rat brain (excluding cerebellum) ([³H]serotonin), rat striatal synaptosomes ([³H]dopamine) or into rat cortical synaptosomes [³H]noradrenalin. The dissected rat brain regions were homogenized in 0.40 M sucrose supplemented with 1 mM nialamid and centrifuged at 1000×g for 10 min. The supernatants were further centrifuged for 30 min at 20.000×g, 4° C. and resuspended in Krebs-Ringer buffer, pH 7.4 supplemented with 0.2 g/l ascorbic acid. Test compounds and membranes were added in 96 well plates and the incubation was started by adding either 10 nM [³H]serotonin, 12.5 nM [³H]dopamine or 10 nM [³H]noradrenalin for 15 min at 37° C. except for [³ H]dopamine uptake (5 min at 20° C.). Non-specific uptake was defined as uptake in the presence of 10 μM citalopram, 100 μM benttropin or 20 μM talsupram, respectively and accounted for 5-10% of total uptake. Samples were filtered over Whatman GF/C filters and the IC₅₀'s were estimated using non-linear regression analysis from at least 8 points dose-response curves with triplicate determinations.

Measurements of high affinity [³H]glutamate uptake into rat brain synaptosomes were performed using homogenized fresh cortex from male Wistar rats (150-200 g) prepared as described above. Synaptosomes (3 mg tissue) were mixed with testcompounds and pre-incubated 5 min at 25° C. The incubation (5 min at 25° C.) was started by adding 50 μl ³H-glutamate (8 nM tracer +0.5 μM L-glutamate) to a final volumen of 1 ml. Samples are filtered directly onto Whatman GF/B glass fiber filters under vacuum and immediately washed with 3×1 ml 0.9% NaCI. The amount of radioactivity on the filters is determined by conventional liquid scintillation counting. Non-specific uptake is determined in triplicate using L-glutamate (1 mM final concentration).

Microdialysis Experiments

Rats (male wistar) were anaesthetized and intracerebral guide cannulas (CMA/12) were stereotaxically implanted into the brain positioning the dialysis probe tip in the ventral hippocampus (co-ordinates 5.6 mm anterior to bregma, lateral −5.0 mm, 7.0 mm ventral to dura). The rats were allowed to recover from surgery for at least 2 days. On the day of the experiment, a microdialysis probe (CMA/12, 0.5 mm diameter, 3 mm length) was inserted through the guide cannula. The probes were connected via a dual channel swivel to a microinjection pump. Perfusion of the microdialysis probe with filtered Ringer solution (145 mM NaCl, 3 mM KCl, 1 mM MgCl₂, 1.2 mM CaCl₂) was begun shortly before insertion of the probe into the brain and continued for the duration of the experiment at a constant flow of 1 μl/min. After 165 min of stabilization, the experiments were initiated. A 20 min sampling regime was used throughout the experimental period. Time points were corrected for lag time of the perfusate from the microdialysis site to the probe outlet. The compound, S-methyl-L-cysteine (Sigma-Aldrich, St. Louis, USA) was dissolved in filtered Ringer solution (in 1 mM concentration) and was locally infused into the ventral hippocampus by reverse dialysis for 140 min (FIG. 1). After the experiments, the rats were sacrificed by decapitation. The brains were removed, frozen and sectioned (20 μm), and the position of the probes was verified. The concentrations of amino acids in the dialysates were analyzed by means of HPLC with fluorescence detection after precolumn online derivatisation with o-phatalaldehyde. The system consisted of a Hypersil AA-ODS column (5 μm, 2.1×200 mm, Agilent) with a Agilent 1100 fluoresence detector (excitation, 266-340 nm; emission, 305-340 nm). Mobile phases consisted of A: 20 mM sodium acetate, 0.018% triethylamine, 0.3% tetrahydrofuran, pH 7.2. B: 20 mM sodium acetate, 40% acetonitrile and 40% methanol, pH 7.2. The oven temperature was set at 40° C. and flow rate was 0.45 ml/min. Data were collected and analysed using ChemStation software (Agilent) after calibration with a range of standard amino acid solutions (0.1-10 μM). The mean value of 3 consecutive samples immediately preceding compound administration served as the basal level for each experiment and data were converted to percentage of basal (mean basal pre-injection values normalized to 100%).

1. A pharmaceutical composition comprising an inhibitor of the asc-1 transporter.

2. A method of treating schizophrenia, psychosis, Parkinson's disease, depression, obsessive compulsive disorder, an anxiety disorder, a bipolar disorder, epilepsy; or memory and attention deficits resulting from Alzheimer's disease, Parkinson's disease, trauma and or stroke, in a human suffering from such a disease comprising administering an effective amount of an inhibitor of the asc-1 transporter to the human.

3. The method of claim 2, wherein said human suffers from schizophrenia.

4. A method of enhancing function of normal or abnormal excitable tissue in a human comprising administering an effective amount of an inhibitor of asc-1 transporter to the human.

5. The method of claim 4, wherein said method results in enhancement of associative learning and memory.

6. The method of claim 2, wherein said treatment is prophylactic.

7. The method of claim 2, wherein said treatment is restorative.

8. A method for identifying compounds that are antagonists of asc-1 mediated D-serine-transport comprising incubating synaptically derived brain membrane fragments (“synaptosomes”) with labeled D- or L-serine, and with a test compound to be tested as a D-serine transport antagonist and thereafter measuring the D- or L-serine uptake in comparison w to a control.

9. The method of claim 8, wherein said labeled D- or L-serine, is radioactively labeled.

10. The method of claim 8, wherein incubating is conducted in the presence of a selective inhibitor of system asc.

11. Compounds identified by the assay of claim 8 as inhibitors of asc-1 mediated transmembrane transport of D-serine.

12. A pharmaceutical composition comprising a non-toxic therapeutically effective amount of an asc-1 inhibitor according to claim 11 and a pharmaceutically acceptable carrier.

13. The pharmaceutical composition of claim 1 in the form of a unit dose, wherein the quantity of inhibitor in a the unit dose is from about 0.1 mg to 1000 mg.

14. The method of claim 4, wherein the treatment is prophylactic.

15. The method of claim 4, wherein the treatment is restorative.

16. The method of claim 10, wherein the selective inhibitor is alanine.

17. The pharmaceutical composition of claim 13, wherein the quantity of inhibitor is from about 1 mg to 300 mg.

18. The pharmaceutical composition of claim 12 in the form of a unit dose, wherein the quantity of inhibitor in the unit dose is from about 0.1 mg to 1000 mg.

19. The pharmaceutical composition of claim 18, wherein the quantity of inhibitor is from about 1 mg to 300 mg.
